(19) United States
(12) Patent Application Publication
(10) Pub. No.: US 2004/0038355 A1
(43) Pub. Date: Feb. 26, 2004

(54) Plate-like magnetic particles

(76) Inventors: Reiner Vogt, Kranichstein (DE); Wilhelm Gobel, Gr. Bieberau (DE); Sabine George, Bensheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/250,402

(22) PCT Filed: Dec. 27, 2001
(86) PCT No.: PCT/EP01/15316

(30) Foreign Application Priority Data
Dec. 30, 2000 (DE) 100 65 761.3

(51) Int. Cl. C12N 9/00; A01N 59/06

(52) U.S. Cl. 435/183; 424/400; 424/684

(57) ABSTRACT

Platelet-shaped multilayered particles wherein the core includes alumina and/or a mixed phase of alumina and silica, the interlayer includes amorphous silica and the sheath includes iron, the iron being present in elemental form and/or in the form of one or more ferriferous compounds selected from the group consisting of ferdisilicide, maghemite, hematite and magnetite. What is disclosed is a preparative process and the use of the particles in the isolation and separation of organic substances or in enzymatic cleavage reactions and in a magnetically stabilized moving bed.

The invention relates to platelet-shaped magnetic particles based on coated alumina particles. The invention further relates to the preparation and use of these particles.

Magnetic particles have diverse applications. For instance, they are used for immobilizing enzymes or separating nucleic acids and proteins.

For instance, U.S. Pat. No. 4,343,901 describes magnetic particles for enzyme immobilization which consist of an inorganic oxide and ferromagnetic particles which are obtained by a sol-gel technique.

U.S. Pat. No. 4,280,918 describes a dispersion of magnetic particles which is manufactured by mixing a dispersion of either Fe₂O₃ or cobalt doped Fe₃O₄ having a positive charge with colloidal SiO₂ having a negative charge and a subsequent ultrasonic treatment.

EP 0 343 934 discloses magnetic particles which consist of a core of a magnetic material and a sheath of an inorganic oxide and are obtained by a sol-gel technique.

DE 196 38 591 discloses spherical magnetic particles based on monodisperse SiO₂ particles spot-coated with magnetic particles of a certain particle size and bearing a covering layer of silica. These particles are used for isolating nucleic acids and biotin and also biotinylated nucleic acids or proteins or other biotin-labelled molecules.

These conventional magnetic particles merely contain magnetic or magnetizable metal oxides and have a correspondingly limited magnetizability.

Moreover, the spherical structure of the abovementioned particles constrains their removability in the isolation methods mentioned and also the surface area of the particles.

It is an object of the present invention to overcome these disadvantages and to provide stable, readily filterable particles possessing high magnetizability and a large surface area.

This object is achieved by providing platelet-shaped multilayered particles whose core includes alumina and/or a mixed phase of alumina and silica, whose interlayer includes amorphous silica and whose sheath includes iron, the iron being present in elemental form and/or in the form of one or more ferriferous compounds selected from the group consisting of ferdisilicide, maghemite, hematite and magnetite.

All particles having this basic structure are in accordance with the invention, although the fraction contributed by each layer may be varied as necessary. For instance, high fractions of the magnetic or magnetizable sheath improve the magnetic properties. It will be appreciated that, when the particles are to possess high stability, the core and the interlayer make a comparatively large contribution to the overall particle.

The invention further provides a preparative process to obtain the layered construction in question.

This process comprises a first step of suspending aluminium powder in water and adding a water-soluble silicate to this suspension at a pH of 5 to 9, preferably 7 to 8, a second step of adding a water-soluble iron salt to the suspension at a pH of 2 to 5, preferably 3 to 4, a third step of removing and optionally washing and drying the particles and a fourth step of calcining the particles. The fourth step is preferably carried out at a temperature of 400 to 800° C., particularly preferably at 500 to 600° C.

The first step comprises the deposition of SiO₂ on the aluminium particle, the second step comprises the deposition of iron hydroxide or iron oxyhydrate and the last step comprises the particles, which now consist of an aluminium core with an SiO₂ interlayer and an iron hydroxide or iron oxyhydrate sheath prior to calcination, being reacted in an aluminothermal process (Thermit process) to form the particles of the invention.

The process may be carried out under a protective gas atmosphere, for example nitrogen, or else under air. In the latter case, the oxidation of the aluminium to alumina can also be effected via elemental oxygen from the air, while only the chemically bonded oxygen of the intermediate and sheath layers is available under a protective gas atmosphere. In any case, however, substantial portions of the oxides, hydroxides or oxyhydrates of the intermediate and sheath layers are reduced to the iron and silicon elements. These can then be present in elemental form side by side or alloyed as ferdisilicide.

Depending on the stoichiometry between the aluminium base and the amounts used of water-soluble silicate (sodium silicate, for example) and iron salt solution and also on account of the fact that calcination can be carried out not only in air but also in nitrogen, oxygen may not be available in a sufficient amount to completely oxidize the elemental aluminium. The individual layers may therefore also include elemental aluminium, although this does not affect the inventive achievement of the objects. But the oxidation of the aluminium is also accompanied by the desired partial reduction of the silica to form elemental silicon, which may likewise be present in the particles without impairing the inventive achievement of the object. Usually, however, the elemental silicon thus formed will be alloyed wholly or partly with the elemental iron obtained in the aluminothermal process, to form ferdisilicide.

By varying the amounts of aluminium, water-soluble silicate and iron salt used in steps 1 and 2, the properties of the particles—as mentioned above—may be varied as desired. Similarly, by varying the composition it is possible to influence the colour of the particles.

The type of atmosphere during the calcining step may also influence the composition of the core in that alumina-silica mixed phases form preferentially under protective gas, for example nitrogen.

It is advantageous for most applications for the core to comprise 10 to 90% by weight, preferably 15 to 40% by weight, of the particle and the coating to comprise 90 to 10% by weight, preferably 85 to 60% by weight. A particularly advantageous fraction for the sheath is a fraction of 5 to 50% by weight, preferably 10 to 45% by weight, based on the overall particle.

In a preferred embodiment, an inorganic and/or organic coupling reagent may be applied subsequently atop the sheath. A possible inorganic coupling agent applied may be a further layer of silica, to which, for example, nucleic acids can bind without further coating. Such a silica layer may in turn be modified with covalently attached organic groups. The silanes used for this are constructed in such a way that they have functional groups with which the material to be separated off may be reversibly bonded to the magnetic particles. Details are discernible from DE 42 33 396 and DE 43 16 814. To recover biotin or biotinylated substances, for example, a possible silanizing agent is γ-aminopropylsilane to which streptavidin is attached.

Inventive particles without aftercoating or else aftercoated with inorganic coupling reagents and/or silanes may also physisorb, chemisorb or covalently attach enzymes, so that the particles have an enzyme-modified surface.

The particles of the invention may accordingly be used for isolating nucleic acids and biotin and also biotinylated proteins from aqueous solutions but also in enzyme reactors.

Furthermore, the particles of the invention may be used in a magnetized stabilized moving bed.

The preparative example hereinbelow is intended to more particularly describe the invention without limiting it.

### PREPARATIVE EXAMPLE

50 g of aluminium powder (Standard Resist 501 from Eckart) are suspended in 2000 ml of water at 75° C. 280 g of an approximately 11% by weight sodium silicate solution are metered in at a pH of 7.5, which is kept constant by concurrent addition of 18% hydrochloric acid. On completion of the sodium silicate addition, the batch is stirred for 1 h and the pH gradually adjusted to 3.5 with hydrochloric acid. During the subsequent addition of 740 ml of an iron(III) chloride solution (iron content: 7%), the pH is kept constant by means of 32% sodium hydroxide solution. Following a supplementary 30 minute stir, the suspension is filtered off with suction, washed and dried at 110° C. overnight. This is followed by calcining under a protective gas at 600° C. for 30 min to obtain the particles of the invention.

Further particles according to the invention were prepared by varying not only the sodium silicate quantity but also the iron(III) chloride quantity.

1. Platelet-shaped particles, characterized in that they possess a plurality of layers, the core including alumina and/or a mixed phase of alumina and silica, the interlayer including amorphous silica and the sheath including iron, the iron being present in elemental form and/or in the form of one or more ferriferous compounds selected from the group consisting of ferdisilicide, maghemite, hematite and magnetite.

2. Platelet-shaped particles according to claim 1, characterized in that atop the sheath there has been applied a further layer comprising inorganic and/or organic coupling reagents capable of binding to nucleic acids or proteins.

3. Platelet-shaped particles according to claim 2, characterized in that the inorganic coupling reagent is SiO₂ and the organic coupling reagent is a specifically functionalized silane, the latter being optionally attached to an enzyme.

4. Platelet-shaped particles according to one or more of the preceding claims, characterized in that the core comprises 10 to 90% by weight, preferably 15 to 40% by weight, of the particle and the coating comprises 90 to 10% by weight, preferably 85 to 60% by weight, the sheath comprising 5 to 50% by weight, preferably 10 to 45% by weight, of the overall particle.

5. Process for preparing platelet-shaped particles, characterized in that it comprises
a first step of suspending aluminium powder in water and adding a water-soluble silicate to this suspension at a pH of 5 to 9, preferably 7 to 8,
a second step of adding a water-soluble iron salt to the suspension at a pH of 2 to 5, preferably 3 to 4,
a third step of removing and optionally washing and drying the particles and
a fourth step of calcining the optionally predried particles.

6. Process according to claim 5, characterized in that the fourth step is carried out at a temperature of 400 to 800° C., preferably 500 to 600° C.

7. Process according to claim 6, characterized in that it comprises a fifth step of aftercoating by addition of a water-soluble silicate as per step 1.

8. Process according to one or more of claims 5 to 7, characterized in that it comprises a further step of aftercoating with a functionalized silane.

9. Process according to claim 8, characterized in that the silane is attached to an enzyme.

10. Platelet-shaped particles, characterized in that they are obtained by a process according to any of claims 5 to 9.

11. Use of the platelet-shaped particles according to one or more of claims 1 to 4 and 10 for isolating nucleic acids and biotin and also biotinylated proteins from aqueous solutions.

12. Use of the platelet-shaped particles according to one or more of claims 1 to 4 and 10 in a magnetized stabilized moving bed.

13. Use of the platelet-shaped particles according to one or more of claims 1 to 4 and 10 in the enzymatic conversion of substrates.
